(19) United States
(12) Patent Application Publication
(10) Pub. No.: US 2018/0055931 A1
(43) Pub. Date: Mar. 1, 2018

(54) ANIMAL CELL REGENERATION SPEED CONTROLLING METHOD AND ANIMAL CELL REGENERATION SPEED CONTROLLER

(71) Applicant: TOYOTA BOSHOKU KABUSHIKI KAISHA, Aichi-ken (JP)

(72) Inventors: Yuko YAMAMOTO, Aichi-ken (JP); Mao MORIMOTO, Aichi-ken (JP)

(73) Assignee: TOYOTA BOSHOKU KABUSHIKI KAISHA, Aichi-ken (JP)

(21) Appl. No.: 15/661,588

(22) Filed: Jul. 27, 2017

(30) Foreign Application Priority Data
Sep. 1, 2016 (JP) 2016-171237

(51) Int. Cl. A61K 41/00; A61H 99/00

(52) U.S. Cl. CPC A61K 41/00; A61H 2201/50; A61H 99/00

(57) ABSTRACT

The present invention is a method for controlling a regeneration speed of an animal cell that includes a step of grasping tide-generating force and a step of giving a physical or chemical stimulus to the animal cell according to the magnitude of variation in the tide-generating force.

### CROSS REFERENCE TO RELATED APPLICATION

The present application claims priority under 35 U.S.C. §119 of Japanese Patent Application No. 2016-171237, filed on Sep. 1, 2016, the disclosure of which is expressly incorporated by reference herein in its entirety.

### BACKGROUND OF THE INVENTION

### Technical Field

The present invention relates to an animal cell regeneration speed controlling method and an animal cell regeneration speed controller, which make it possible to change a metabolism of the animal cell and to control the regeneration speed of the animal cell.

### Related Art

In recent years, studies on regeneration methods intended, for example, to promote a regeneration speed are actively made in a regenerative medical field.

Systems, methods, devices and the like for promoting the growth of a regenerating tissue from a wound surface of a wound area in a biological tissue structure of a living human or animal body into the wound area in a predetermined direction are known as a specific technique for promoting a regeneration of a living tissue (see JP-A 2000-510712).

In the regeneration promoting methods according to JP-A 2000-510712, a growing direction of cell tissues is fixed by a mold or local drug concentration adjustment, resulting in promotion of regeneration. Therefore, such methods require a mold or local drug concentration adjustment for fixing the growing direction for each target tissue, and thus are disadvantageously apt to involve very complicated procedures and require a lot of time and efforts. Techniques that can promote the regeneration of cell tissues by an easier method are demanded.

### SUMMARY OF THE INVENTION

An object of the present invention is to provide an animal cell regeneration speed controlling method and an animal cell regeneration speed controller, that can change a metabolism of the animal cell and can easily control the regeneration speed of the animal cell.

The present inventor has found, through intensive and deliberate studies, that the magnitude of variation in tide-generating force unexpectedly acts on the regeneration of an animal cell, and that a physical or chemical stimulus is given to the animal cell according to the magnitude of the variation, thereby allowing a change in metabolism of the animal cell; and has finally achieved the present invention.

In order to solve the above problem, the invention claimed in claim 1 relates to an animal cell regeneration speed controlling method for controlling the regeneration speed of an animal cell, the method comprising:

grasping tide-generating force; and

giving a physical or chemical stimulus to the animal cell according to magnitude of variation in tide-generating force.

The invention claimed in claim 2 relates to the animal cell regeneration speed controlling method according to claim 1, wherein at least one of relative gravity acceleration, distance from the center of the earth to an implementation spot, name of tide, difference in celestial longitude, and lunar age is used as an index of the tide-generating force.

The invention claimed in claim 3 relates to the animal cell regeneration speed controlling method according to claim 1, wherein the stimulus is given at the timing of a spring tide.

The invention claimed in claim 4 relates to the animal cell regeneration speed controlling method according to claim 2, wherein the stimulus is given at the timing of a spring tide.

The invention claimed in claim 5 relates to the animal cell regeneration speed controlling method according to claim 1, wherein the stimulus is given at the timing of a neap tide.

The invention claimed in claim 6 relates to the animal cell regeneration speed controlling method according to claim 2, wherein the stimulus is given at the timing of a neap tide.

In order to solve the above problem, the invention claimed in claim 7 relates to an animal cell regeneration speed controller for controlling the regeneration speed of an animal cell, the controller comprising:

a tide-generating force grasping means of grasping tide-generating force; and

a stimulus controlling means of controlling a physical or chemical stimulus to be given to the animal cell according to the magnitude of variation in the tide-generating force.

The invention claimed in claim 8 relates to the animal cell regeneration speed controller according to claim 7, wherein at least one of relative gravity acceleration, distance from the center of the earth to an implementation spot, name of tide, difference in celestial longitude, and lunar age is used as an index of the tide-generating force in the tide-generating force grasping means.

The invention claimed in claim 9 relates to the animal cell regeneration speed controller according to claim 7, wherein the stimulus is given at the timing of a spring tide in the stimulus controlling means.

The invention claimed in claim 10 relates to the animal cell regeneration speed controller according to claim 8, wherein the stimulus is given at the timing of a spring tide in the stimulus controlling means.

The invention claimed in claim 11 relates to the animal cell regeneration speed controller according to claim 7, wherein the stimulus is given at the timing of a neap tide in the stimulus controlling means.

The invention claimed in claim 12 relates to the animal cell regeneration speed controller according to claim 8, wherein the stimulus is given at the timing of a neap tide in the stimulus controlling means.

In the present invention, the animal cell is, for example, a cell of mammals, birds, reptiles, amphibians, fish, or invertebrates such as poriferans, cnidarians, flatworms, mollusks, annelids, echinoderms and arthropod.

### Effect of the Invention

According to the animal cell regeneration speed controlling method of the present invention, the metabolism of animal cells can be easily changed according to the magnitude of variation in tide-generating force to easily control the regeneration speed of the animal cells. That is to say, the regeneration speed of the animal cells can be promoted or suppressed according to an intended use. For example, when the regeneration of the animal cells is promoted, it is possible to improve the regeneration efficiency and shorten the regeneration period. Therefore, the regeneration cost can be reduced.

In the case where at least one of relative gravity acceleration, distance from the center of the earth to an implementation spot, name of tide, difference in celestial longitude, and lunar age is used as an index of the tide-generating force, the tide-generating force may be easily grasped eliminating the necessity for special facilities and the metabolism of the animal cell can be effectively changed.

Further, when a physical or chemical stimulus is given to an animal cell at the timing of a spring tide in this regeneration speed controlling method, the metabolism of the animal cell can be sufficiently changed. Especially, the regeneration speed of the animal cell can be promoted.

Also, when a physical or chemical stimulus is given to an animal cell at the timing of a neap tide in this regeneration speed controlling method, the metabolism of the animal cell can be sufficiently changed. Especially, the regeneration speed of the animal cell can be suppressed, and this method can be applied, for example, to the suppression of cancer cells.

According to the animal cell regeneration speed controller of the present invention, the metabolism of animal cells can be easily changed according to the magnitude of variation in tide-generating force to easily control the regeneration speed of the animal cells. That is to say, the regeneration speed of the animal cells can be promoted or suppressed according to an intended use. For example, when the regeneration of the animal cells is promoted, it is possible to improve the regeneration efficiency and shorten the regeneration period. Therefore, the regeneration cost can be reduced.

In the case where at least one of relative gravity acceleration, distance from the center of the earth to an implementation spot, name of tide, difference in celestial longitude, and lunar age is used as an index of the tide-generating force in the tide-generating force grasping means, the tide-generating force may be easily grasped and the metabolism of the animal cell can be effectively changed.

In the case where the stimulus is given at the timing of a spring tide in the stimulus controlling means, the metabolism of the animal cell can be sufficiently changed. In particular, the regeneration speed of the animal cell can be promoted.

In the case where the stimulus is given at the timing of a neap tide in the stimulus controlling means, the metabolism of the animal cell can be sufficiently changed. In particular, the regeneration speed of the animal cell can be suppressed and the method can also be applied, for example, to suppress cancer cells.

### BRIEF DESCRIPTION OF THE DRAWINGS

FIG. 1 is a graph indicating a temporal change in relative gravity acceleration when the standard gravity acceleration (1G) is defined as a reference (zero point); and

FIG. 2 is a schematic diagram for explaining the cutting position of a planarian.

### DESCRIPTION OF THE EMBODIMENTS

### 1. Animal Cell Regeneration Speed Controlling Method

The animal cell regeneration speed controlling method of the present invention is characterized in including a step of grasping tide-generating force and a step of giving a physical or chemical stimulus to the animal cell according to the magnitude of variation in the tide-generating force.

The above tide-generating force can be indicated using, as an index, at least one of relative gravity acceleration (theoretical value), weather data (e.g., atmospheric pressure, name of tide, tidal level and difference in tidal level), difference in celestial longitude (difference in celestial longitude between the sun and the moon), lunar age and distance from the center of the earth to an implementation spot.

Among these, in the regeneration speed controlling method of the present invention, at least one of relative gravity acceleration, distance from the center of the earth to an implementation spot, name of tide, difference in celestial longitude, and lunar age is preferably used as an index of the tide-generating force from the viewpoint of easiness of prediction of the magnitude of variation in tide-generating force and the cycle thereof. Especially, the relative gravity acceleration and the distance from the center of the earth to an implementation spot, name of tide, difference in celestial longitude or lunar age are preferably used in combination.

The relative gravity acceleration (RGA) means a relative value of gravity acceleration based on the standard gravity acceleration (1G=9.80665×10⁸ μGal) as a reference (zero point).

The relative gravity acceleration can be calculated by utilizing a commonly-publicized solid tidal force prediction program. Specifically, the relative gravity acceleration at a target spot and the temporal change thereof can be calculated by inputting information on the position of an implementation site (latitude and longitude), date (year, month and day) and time in the solid tidal force prediction program.

As the solid tidal force prediction program, the tide prediction system “GOTIC2” (http://www.miz.nao.ac.jp/staffs/nao99/) or the like can be used.

When this relative gravity acceleration is used as an index of the tide-generating force, the relative gravity acceleration in a predetermined period and a temporal change thereof are predicted, and the magnitude of variation in tide-generating force [for example, difference between the maximum and minimum values (maximum displacement) and total amount of variation (total amount of displacement)] can be obtained from the predictive data.

Incidentally, the duration of the prediction period is not especially limited, and is appropriately adjusted according to need. Specifically, the duration of the prediction period can be defined, for example, as 6 hours, 12 hours, 24 hours, 48 hours, 72 hours, 1 week, 2 weeks, 4 weeks, 3 months, 6 months or 1 year.

A name of tide conforming to the system of the Japan Meteorological Agency or MIRC (Marine Information Research Center) can be utilized as the above name of tide. It can be considered that the magnitude of variation in tide-generating force becomes larger in the order of the timings of neap tide, nagashio, wakashio, nakashio and spring tide.

Incidentally, the difference in celestial longitude is utilized for determination of the name of tide. However, the speed of tidal waves is decreased on the coast of land, and thus the center of the timing of spring tide does not necessarily coincide with the timing when the difference in celestial longitude is 0° (360°) or 180° which will be explained later, and tends to be delayed according to the latitude and/or land form. This tendency also applies to the center of the timing of neap tide. Therefore, this delay is defined as 12 degrees for determination of the name of tide in the system of the Japan Meteorological Agency, and, on the other hand, is defined as 7 degrees for determination thereof in the system of MIRC.

On this index, the predictive data in a predetermined period ranging from short to long can be readily obtained, for example, from the data offered by the Japan Meteorological Agency (http://www.jma.go.jp/jma/index/html) and the data offered by MIRC.

When the difference in celestial longitude is used as an index of the tide-generating force, the magnitude of variation in tide-generating force can be theoretically considered to be larger at a timing when the difference in celestial longitude is closer to 0° (360°) or 180° and to be smaller at a timing when the difference in celestial longitude is closer to 90° or 270° in the cycle of the difference in celestial longitude (0° to 360°).

On this index, the predictive data in a predetermined period ranging from short to long can be readily obtained, for example, from the Ephemeris Computation Office, NAOJ (http://eco.mtk.nao.ac.jp/koyomi/).

When the lunar age is used as an index of the tide-generating force, the magnitude of variation in tide-generating force can be theoretically considered to be larger at a timing when the lunar age is closer to 0 (30) or 15.0 and to be smaller at a timing when the lunar age is closer to 7.5 or 22.5 in the cycle of the lunar age (0 to 30).

On this index, the predictive data in a predetermined period ranging from short to long can be readily obtained, for example, from the Ephemeris Computation Office, NAOJ (http://eco.mtk.nao.ac.jp/koyomi/) and a common lunar calendar.

When the distance from the center of the earth to an implementation spot is used as an index, the magnitude of variation in tide-generating force can be theoretically considered to be larger when the change of the distance per unit time is larger and to be smaller when the change of the distance per unit time is smaller.

On this index, the predictive data in a predetermined period ranging from short to long can be readily obtained, for example, from the Ephemeris Computation Office, NAOJ (http://eco.mtk.nao.ac.jp/koyomi/).

The above animal cell in the present invention is not especially limited, and specifically includes animal cells of mammals, birds, reptiles, amphibians, fish and invertebrates (poriferans, cnidarians, flatworms, mollusks, annelids, echinoderms, arthropod and the like).

Examples of the physical stimulus to the animal cell include stimuli generated by cutting, piercing, vibration, pressure, tension, heat, light, acoustic wave, acceleration and electricity.

Examples of the chemical stimulus to the animal cell include stimuli generated by contact or administration of a chemical such as a drug.

Examples of the drug include substances having an action of promoting cell division such as substances having mitogen activity, plant lectins, plant hormones, Vitamin A derivatives, and molecular target agents which act on the cell division signal pathway; substances having the action of suppressing cell division such as anticancer agents, plant alkaloids and molecular target agents which act on the cell division signal pathway; and the like.

The physical stimulus and chemical stimulus to the animal cell may be combined with each other. The physical stimulus or chemical stimulus may be either a direct stimulus to an animal cell or an indirect stimulus, for example, to a living body surface having the cell.

Incidentally, the stimulus imparting conditions such as the type of stimulus, amount thereof, number of times of stimulation and stimulation time are appropriately adjusted depending, for example, on the kind of animal cell to which the stimulus is to be given.

In the present invention, the timing of imparting the above stimulus is determined according to the magnitude of variation in tide-generating force as described above.

Specifically, the manner of imparting the stimulus includes: (1) a manner of giving a physical or chemical stimulus to an animal cell at a timing when the magnitude of variation in tide-generating force is relatively large (for example, at the timing of a spring tide), for example, based on the grasped predictive data on tide-generating force; and (2) a manner of giving a physical or chemical stimulus to an animal cell at a timing when the magnitude of variation in tide-generating force is relatively small (for example, at the timing of a neap tide), for example, based on the grasped predictive data on tide-generating force.

In the regeneration speed controlling method of the present invention, when a physical or chemical stimulus is given to an animal cell at a timing when the variation in tide-generating force is large as in the above manner (1), the metabolism of the animal cell can be sufficiently changed. Especially, the regeneration speed of the animal cell can be promoted.

On the other hand, in the regeneration speed controlling method of the present invention, when a physical or chemical stimulus is given to an animal cell at a timing when the variation in tide-generating force is small as in the above manner (2), the metabolism of the animal cell can be sufficiently changed. Especially, the regeneration speed of the animal cell can be suppressed, and this method can be applied, for example, to the suppression of cancer cells.

Also, in the present invention, the timing of imparting a stimulus can be determined for the variations in tide-generating force in specific periods such as the following periods (a) to (d), for example, based on the grasped predictive data on tide-generating force:

(a) a period until the elapse of a certain period (for example, 6 hours, 12 hours, 24 hours, 2 days, 3 days or 1 week) immediately after impartation of a stimulus;

(b) a period until the timing of beginning of stem cell division due to a stimulus immediately after the impartation of the stimulus;

(c) a period until the timing of beginning of stem cell differentiation due to a stimulus immediately after the impartation of the stimulus; and

(d) a period until the timing of completion of stem cell differentiation due to a stimulus immediately after the impartation of the stimulus.

When a physical or chemical stimulus is given to an animal cell at a timing when the variation in tide-generating force for each of the timings (a) to (d) is relatively large, the metabolism of the animal cell can be sufficiently changed. Especially, the regeneration speed of the animal cell can be promoted.

Also, when a physical or chemical stimulus is given to an animal cell at a timing when the variation in tide-generating force for each of the timings (a) to (d) is relatively small, the metabolism of the animal cell can be sufficiently changed. Especially, the regeneration speed of the animal cell can be suppressed, and this method can be applied, for example, to the suppression of cancer cells.

Incidentally, the timing of beginning of stem cell division, timing of beginning of stem cell differentiation and timing of completion of stem cell differentiation each vary depending on the species of the target animal.

### 2. Animal Cell Regeneration Speed Controller

The animal cell regeneration speed controller of the present invention is intended for controlling the regeneration speed of an animal cell, and is characterized in including a tide-generating force grasping means, and a stimulus controlling means of controlling a physical stimulus or chemical stimulus to be given to the animal cell according to the magnitude of variation in the tide-generating force.

Examples of the above tide-generating force grasping means include a means of calculating the relative gravity acceleration to grasp the tide-generating force, a means of obtaining weather data (atmospheric pressure, name of tide, tidal level, difference in tidal level, etc.) to grasp the tide-generating force, a means of calculating or obtaining the difference in celestial longitude (difference in celestial longitude between the sun and the moon) to grasp the tide-generating force, a means of grasping the tide-generating force from the lunar calendar and a means of calculating the distance from the center of the earth to an implementation spot to grasp the tide-generating force. Among these means, a means of using, as an index, at least one of the relative gravity acceleration, distance from the center of the earth to an implementation spot, name of tide, difference in celestial longitude, and lunar age is preferred.

In the above stimulus controlling means, the conditions for imparting a physical or chemical stimulus to an animal cell are controlled according to the magnitude of variation in tide-generating force (for example, predictive data obtained by the above tide-generating force grasping means).

Examples of the manner of controlling the stimulus impartation according to the magnitude of variation in tide-generating force include: (1) a manner of giving a physical or chemical stimulus to an animal cell at a timing when the magnitude of variation in tide-generating force is relatively large (for example, at the timing of a spring tide), for example, based on the grasped predictive data on tide-generating force; and (2) a manner of giving a physical or chemical stimulus to an animal cell at a timing when the magnitude of variation in tide-generating force is relatively small (for example, at the timing of a neap tide), for example, based on the grasped predictive data on tide-generating force.

Further, there can be indicated a manner of determining the timing of imparting a stimulus for the variations in tide-generating force in specific periods such as the following periods (a) to (d), for example, based on the predictive data on tide-generating force grasped by the above tide-generating force grasping means:

(a) a period until the elapse of a certain period (for example, 6 hours, 12 hours, 24 hours, 2 days, 3 days or 1 week) immediately after impartation of a stimulus;

(b) a period until the timing of beginning of stem cell division due to a stimulus immediately after the impartation of the stimulus;

(c) a period until the timing of beginning of stem cell differentiation due to a stimulus immediately after the impartation of the stimulus; and

(d) a period until the timing of completion of stem cell differentiation due to a stimulus immediately after the impartation of the stimulus.

In the above stimulus controlling means, the stimulus imparting conditions such as the type of stimulus, amount thereof, number of times of stimulation and stimulation time are controlled depending, for example, on the kind of the target animal cell.

### 3. Industrial Applicability

According to the animal cell regeneration speed controlling method and the animal cell regeneration speed controller in the present invention, the metabolism of animal cells can be changed according to the magnitude of variation in tide-generating force to easily control the regeneration speed of the animal cells (for example, promotion of regeneration). Therefore, the animal cell regeneration speed controlling method and the animal cell regeneration speed controller can be widely utilized in various fields associated with regeneration of animal cells (especially, regenerative medical field).

### EXAMPLES

Hereinafter, the present invention will be specifically described using Examples. In the following Examples 1 and 2, regeneration experiment was conducted using Planarians (Dugesia japonica) as one kind of flatworm.

### Example 1 (Animal Cell Regeneration Experiment (i))

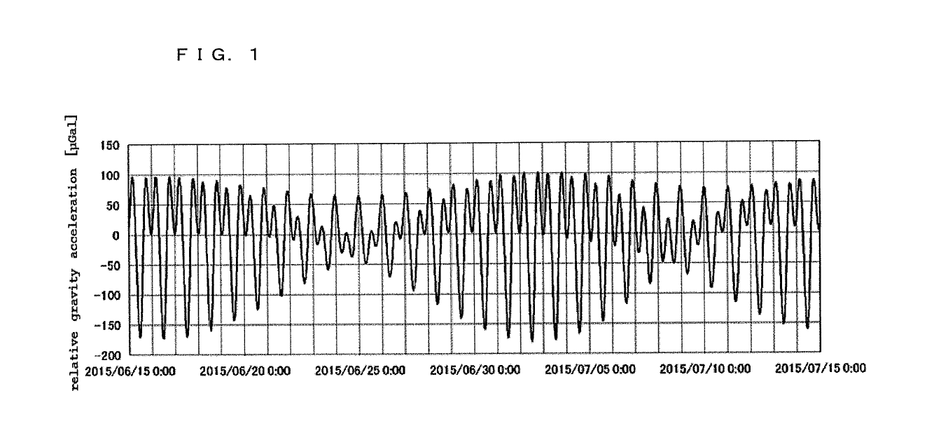

Specifically, the tide prediction system “GOTIC2” (http://www.miz.nao.ac.jp/staffs/nao99/) was firstly used to input the latitude and longitude of an implementation site (Kariya-shi, Aichi Prefecture) to grasp a temporal change in relative value of gravity acceleration [relative gravity acceleration (RGA)] in a predetermined period [one month from June 15 (0 o'clock) to July 15 (23 o'clock) in 2015] at an experiment place (see FIG. 1). Then, the timing of a spring tide with a large variation in tide-generating force and the timing of a neap tide with a small variation in tide-generating force in the period were extracted (spring tide: June 30; neap tide: July 8; and system: system of the Japan Meteorological Agency). Planarians were cut, and the timing of carrying out the eye regeneration experiment was determined.

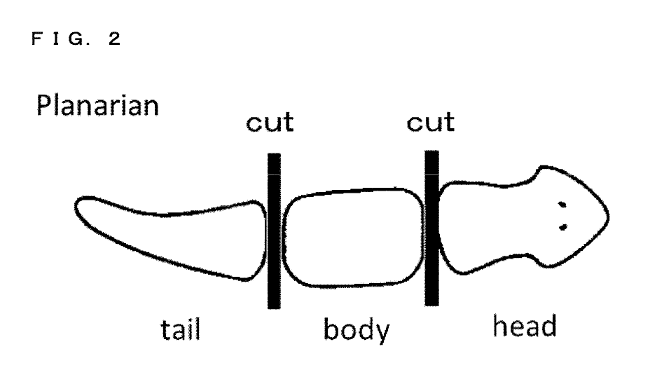

On the day of spring tide indicated above, 50 planarians having an equivalent size were selected from a group of planarians bred under the same conditions, and classified as Group A (n=50). Thereafter, the living bodies were cut at two places to be divided into three parts (head, body and tail) (see FIG. 2). The bodies of Group A were bred in a thermostat bath set to a temperature of 13° C. for 168 hours (7 days) under a condition and in an environment such that water was changed every three days.

Also, on the day of neap tide indicated above, 50 planarians having a size equivalent to that of Group A were selected from a group of planarians bred under conditions similar to those for Group A and classified as Group B (n=50). Then, the bodies of Group B were bred for 168 hours (7 days) in the same manner as for Group A.

After growth for the above period, Groups A and B were respectively formalin-fixed, and the degree of regeneration of their eyes was confirmed with an optical microscope to determine the regeneration degree based on the following three references (incidentally, as for the references for the degree of regeneration, see Teresa Adel et al., Dev Genes Evol (2008) 218:89-103). The planarians ranked as “regeneration degree 3” were determined to “have been completely regenerated.” The number of the planarians determined to “have been completely regenerated” was counted, and the result thereof is indicated in Table 1.

“Regeneration degree 1”: Eyes being developed cannot be identified (unregenerated).

“Regeneration degree 2”: Eyes being developed can be identified (insufficiently regenerated).

“Regeneration degree 3”: Eyes can be identified (completely regenerated).

Incidentally, Table 1 also indicates the maximum and minimum values and maximum amount of variation [difference between the maximum and minimum values (maximum displacement)] of RGA in the temporal change in RGA in a period until 12 hours after cutting treatment [temporal change in RGA (μGal) based on the standard gravity acceleration (1G) as a reference (zero point)].

TABLE 1

| | Group A | Group B |
|---|---|---|
| Timing of cutting treatment (name of tide) | spring tide | neap tide |
| Maximum value of RGA (μGal) | 88.1 | 78.2 |
| Minimum value of RGA (μGal) | −158.2 | −50.0 |
| Maximum amount of variation in RGA (μGal) | 246.3 | 128.2 |
| Number of completely regenerated | 23 | 16 |
| individuals (individuals) (n = 50) | | |

As indicated in Table 1, in Group A subjected to cutting treatment at the timing of spring tide, 23 individuals (46% of the total) were determined to “have been completely regenerated” after 168 hours had elapsed since cutting.

Contrary to this, in Group B subjected to cutting treatment at the timing of neap tide, 16 individuals (32% of the total) were determined to “have been completely regenerated” after 168 hours had elapsed since cutting.

At this time, the maximum amount of variation in RGA in a period until 12 hours after cutting in Group A was 246.3 μGal, showing an experience difference of 118.1 μGal from 128.2 μGal which is the maximum amount of variation in RGA in Group B.

As a result of this, it could be confirmed that Group A subjected to cutting treatment at the timing of spring tide with a large variation in tide-generating force included more individuals determined to “have been completely regenerated” than Group B subjected to cutting treatment at the timing of neap tide with a small variation in tide-generating force, and that the eye regeneration speed of Group A was increased more than that of Group B.

### Example 2 (Animal Cell Regeneration Experiment (ii))

Planarians (Dugesia japonica), as one kind of flatworm, were used to carry out a functional recovery experiment [functions: brain and eyes (eyesight)] utilizing the fact that they show negative phototaxis (action of avoiding light).

Specifically, the tide prediction system “GOTIC2” (http://www.miz.nao.ac.jp/staffs/nao99/) was firstly used to input the latitude and longitude of an implementation site (Kariya-shi, Aichi Prefecture) to grasp a temporal change in relative value of gravity acceleration (RGA) in a predetermined period [September 1 (0 o'clock) in 2015 to August 31 (23 o'clock) in 2016] at an experiment place. Further, the names of tide (according to the system of the Japan Meteorological Agency) in the same period were grasped. Incidentally, the average value of the difference between the maximum and minimum relative gravity acceleration values during a single day on all days of spring tide was 248.05 μGal (standard deviation: 33.22), in the above predictive data for one year. Also, the average value of the difference between the maximum and minimum relative gravity acceleration values during a single day on all days of neap tide was 133.06 μGal (standard deviation: 15.74).

Next, a plurality of timings of spring tides with a large variation in tide-generating force and timings of neap tides with a small variation in tide-generating force in the above period were extracted [spring tides: (a) March 9, (b) March 23 and (c) April 7; neap tides: (a) March 2, (b) March 16 and (c) April 1; and system: system of the Japan Meteorological Agency]. Planarians were cut, and the day for carrying out the functional recovery experiment was determined.

On the day of spring tide (a) indicated above, 50 planarians having an equivalent size were selected from a group of planarians bred under the same conditions, and classified as Group C (n=50). Thereafter, the living bodies were cut at two places to be divided into three parts (head, body and tail) (see FIG. 2). The bodies (Group C-1) and tails (Group (C-2) of Group C were respectively bred in a thermostat bath set to a temperature of 13° C. for 192 hours (8 days) under a condition and in an environment such that water was changed every three days. Further, also on the days of spring tides (b) and (c) indicated above, the bodies and tails of the planarians subjected to cutting treatment were bred for 192 hours (8 days) in the same manner as for the day of spring tide (a) indicated above.

Also on the day of neap tide (a) indicated above, 50 planarians having a size equivalent to that of Group C were selected from a group of planarians bred under conditions similar to those for the above Group C and classified as Group D (n=50). Thereafter, the bodies (Group D-1) and tails (Group D-2) of Group D were respectively bred for 192 hours (8 days) in the same manner as for Group C. Further, also on the days of neap tides (b) and (c) indicated above, the bodies and tails of the planarians subjected to cutting treatment were bred for 192 hours (8 days) in the same manner as on the day of neap tide (a) indicated above.

The degree of functional recovery of the brain and eyes were confirmed through the following test after 5 days, 6 days, 7 days and 8 days had respectively elapsed since cutting treatment. Incidentally, this test was carried out in five separate parts each for 10 planarians in the respective groups (n=50).

### <Confirmative Test on Functional Recovery>

The planarians subjected to cutting treatment were allowed to stand at a center part (within a region of a circle having a radius of 3 mm from the center point) of a container with suppressed scattering of light [dimensions: 100 in length×140 in width×15 in height (mm); temperature: 13° C.; and water amount: 40 mL], and the center part was irradiated with light (illuminance: 62.5 lux; and distance from the water surface: 36 mm) for 3 minutes in a dark place to check the positions of the planarians after irradiation. Then, the positions of the respective planarians after irradiation were recorded, and the moving distances from the center were measured. The average moving distances (mm) are indicated in Tables 2 and 3.

Incidentally, this test utilizes the negative phototaxis of planarians, and it is considered that, if they can take the action of avoiding a light stimulus, the functions of the brain and eyes have recovered.

Also, Tables 2 and 3 also indicate the maximum and minimum values and maximum amount of variation [difference between the maximum and minimum values (maximum displacement)] of RGA in the temporal change in RGA in a period until 12 hours after cutting treatment [temporal change in RGA (μGal) based on the standard gravity acceleration (1G) as a reference (zero point)].

TABLE 2

| | | Group C-1 (regenerated from body) | | | Group D-1 (regenerated from body) | | |
|---|---|---|---|---|---|---|---|
| Timing of cutting treatment | | spring tide | spring tide | spring tide | neap tide | neap tide | neap tide |
| (name of tide) | | (a) | (b) | (c) | (a) | (b) | (c) |
| Maximum value of RGA (μGal) | | 113.5 | 90.8 | 109.9 | 32.5 | 51.9 | 55.8 |
| Minimum value of RGA (μGal) | | −89.4 | −86.5 | −128.8 | −75.5 | −92.3 | −84.7 |
| Maximum amount of variation | | 202.9 | 177.2 | 238.7 | 108.0 | 144.3 | 140.4 |
| in RGA (μGal) | | | | | | | |
| Average | After breeding | 27.3 | — | 20.6 | — | 16.3 | 21.3 |
| moving | for 5 days | Average (n = 100); 24.0 | | | Average (n = 100); 18.8 | | |
| distance (mm) | After breeding | 29.0 | 21.4 | 24.3 | 24.4 | 15.4 | — |
| (n = 50) | for 6 days | Average (n = 150); 24.9 | | | Average (n = 100); 19.9 | | |
| | After breeding | 34.3 | 27.9 | 26.1 | 27.2 | 19.7 | 23.6 |
| | for 7 days | Average (n = 150); 29.4 | | | Average (n = 150); 23.4 | | |
| | After breeding | 26.8 | 27.6 | 27.9 | 28.7 | 21.9 | — |

| | | |
|---|---|---|
| for 8 days | Average (n =150); 27.4 | Average (n = 100); 25.3 |

TABLE 3

| | | Group C-2 (regenerated from body) | | | Group D-2 (regenerated from body) | | |
|---|---|---|---|---|---|---|---|
| Timing of cutting treatment | | spring tide | spring tide | spring tide | neap tide | neap tide | neap tide |
| (name of tide) | | (a) | (b) | (c) | (a) | (b) | (c) |
| Maximum value of RGA (μGal) | | 113.5 | 90.8 | 109.9 | 32.5 | 51.9 | 55.8 |
| Minimum value of RGA (μGal) | | −89.4 | −86.5 | −128.8 | −75.5 | −92.3 | −84.7 |
| Maximum amount of variation | | 202.9 | 177.2 | 238.7 | 108.0 | 144.3 | 140.4 |
| in RGA (μGal) | | | | | | | |
| Average | After breeding | 19.9 | — | 20.5 | — | 14.8 | 22.0 |
| moving | for 5 days | Average (n = 100); 20.2 | | | Average (n = 100); 18.4 | | |
| distance (mm) | After breeding | 21.2 | 20.5 | 26.0 | 17.9 | 17.7 | — |
| (n = 50) | for 6 days | Average (n = 150); 22.6 | | | Average (n = 100); 17.8 | | |
| | After breeding | 24.8 | 29.1 | 32.2 | 19.2 | 23.1 | 21.3 |
| | for 7 days | Average (n = 150); 28.7 | | | Average (n = 150); 21.2 | | |
| | After breeding | 27.9 | 26.2 | 29.3 | 22.4 | 21.3 | — |

| | | |
|---|---|---|
| for 8 days | Average (n = 150); 27.8 | Average (n = 100); 21.9 |

As indicated in Tables 2 and 3, the average moving distances after breeding for 5 to 8 days (n=150 or 100) for Group C-1 (regenerated from the body) subjected to cutting treatment at the timings of spring tides (a) to (c) were 24.0 mm (after breeding for 5 days), 24.9 mm (after breeding for 6 days), 29.4 mm (after breeding for 7 days) and 27.4 mm (after breeding for 8 days), respectively. Also, the average moving distances after breeding for 5 to 8 days (n=150 or 100) for Group C-2 (regenerated from the tail) subjected to cutting treatment at the timings of spring tides (a) to (c) were 20.2 mm (after breeding for 5 days), 22.6 mm (after breeding for 6 days), 28.7 mm (after breeding for 7 days) and 27.8 mm (after breeding for 8 days), respectively.

Contrary to this, the average moving distances after breeding for 5 to 8 days (n=150 or 100) for Group D-1 (regenerated from the body) subjected to cutting treatment at the timings of neap tides (a) to (c) were 18.8 mm (after breeding for 5 days), 19.9 mm (after breeding for 6 days), 23.4 mm (after breeding for 7 days) and 25.3 mm (after breeding for 8 days), respectively. Also, the average moving distances after breeding for 5 to 8 days (n=150 or 100) for Group D-2 (regenerated from the tail) subjected to cutting treatment at the timings of neap tides (a) to (c) were 18.4 mm (after breeding for 5 days), 17.8 mm (after breeding for 6 days), 21.2 mm (after breeding for 7 days) and 21.9 mm (after breeding for 8 days), respectively.

As a result of this, it could be confirmed that Groups C-1 (regenerated from the body) and C-2 (regenerated from the tail) subjected to cutting treatment at the timings of spring tides with a large variation in tide-generating force exhibited longer average moving distances after breeding for 5 to 8 days, respectively, than Groups D1 (regenerated from the body) and D-2 (regenerated from the tail) subjected to cutting treatment at the timings of neap tides with a small variation in tide-generating force, and that Group C exhibited an increased functional recovery speed by about 1 to 2 day(s) as compared with Group D. Incidentally, these pieces of data could be confirmed to show 5% significance also in the statistical processing (Student's t-test).

From the results of Examples 1 and 2 presented above, it is considered that the degree of an influence of stimulus impartation (cutting treatment) on the metabolism of an animal cell varies depending on the magnitude of variation in tide-generating force at the timing of imparting a stimulus, and that the magnitude of the variation has an influence also on the regeneration speed of the cell. In brief, the magnitude of the variation is considered to change the state of the cell and has an influence on the cell growth.

Especially, it can be inferred that the magnitude of variation in tide-generating force in a period until the timing of beginning of stem cell division, a period until the timing of beginning of stem cell differentiation or a period until the timing of completion of stem cell differentiation due to impartation of a stimulus would have a strong influence.

Therefore, a physical stimulus such as cutting treatment is given to an animal cell according to the magnitude of variation in tide-generating force, thereby making it possible to change the metabolism of the animal cell and to control, especially, improve the regeneration speed. Further, it is possible to promote the regeneration speed and improve the regeneration efficiency, thereby shortening the regeneration period and suppressing the regeneration cost.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular structures, materials and embodiments, the present invention is not intended to be limited to the particulars disclosed herein; rather, the present invention extends to all functionally equivalent structures, methods and uses, such as are within the scope of the appended claims.

The present invention is not limited to the embodiments described in detail above, and can be variously modified or changed within the scope presented in the claims of the present invention.

For example, a physical stimulus, cutting treatment, is given in the above Examples, but this stimulus may be a chemical stimulus such as drug administration. Especially, in the case of drug administration, the timing of administering a drug is adjusted according to the magnitude of variation in tide-generating force without adjustment of the drug concentration, thereby making it possible to control the differentiation/division speed and metabolism of the animal cell. Specifically, it is considered that, when the drug effect of suppressing malignant cell growth is enhanced during culture of an animal cell, for example, in regenerative medicine, the suppressive effect can be enhanced by adjusting the timing of administration, not by increasing the drug concentration as conventionally done. It is considered that, as a result, the side effect of the drug suppressing malignant cell growth can be suppressed, and that the growth efficiency of cells desired to increase finally can be improved. Also, it is considered to be possible to reduce the amount of the drug used and to suppress the regeneration cost. Further, it is considered that the number of complicated steps, for example, of adjusting the drug concentration can be reduced.

What is claimed is:

1. An animal cell regeneration speed controlling method comprising:
grasping a tide-generating force; and
giving a physical or chemical stimulus to the animal cell according to the magnitude of variation in the tide-generating force.

2. The animal cell regeneration speed controlling method according to claim 1,
wherein at least one of relative gravity acceleration, distance from the center of the earth to an implementation spot, name of tide, difference in celestial longitude, and lunar age is used as an index of the tide-generating force.

3. The animal cell regeneration speed controlling method according to claim 1,
wherein the stimulus is given at the timing of a spring tide.

4. The animal cell regeneration speed controlling method according to claim 2,
wherein the stimulus is given at the timing of a spring tide.

5. The animal cell regeneration speed controlling method according to claim 1,
wherein the stimulus is given at the timing of a neap tide.

6. The animal cell regeneration speed controlling method according to claim 2,
wherein the stimulus is given at the timing of a neap tide.

7. An animal cell regeneration speed controller comprising,
a tide-generating force grasping means of grasping a tide-generating force; and
a stimulus controlling means of controlling a physical or chemical stimulus to be given to the animal cell according to the magnitude of variation in the tide-generating force.

8. The animal cell regeneration speed controller according to claim 7,
wherein at least one of relative gravity acceleration, distance from the center of the earth to an implementation spot, name of tide, difference in celestial longitude, and lunar age is used as an index of the tide-generating force in the tide-generating force grasping means.

9. The animal cell regeneration speed controller according to claim 7,
wherein the stimulus is given at the timing of a spring tide in the stimulus controlling means.

10. The animal cell regeneration speed controller according to claim 8,
wherein the stimulus is given at the timing of a spring tide in the stimulus controlling means.

11. The animal cell regeneration speed controller according to claim 7,
wherein the stimulus is given at the timing of a neap tide in the stimulus controlling means.

12. The animal cell regeneration speed controller according to claim 8,
wherein the stimulus is given at the timing of a neap tide in the stimulus controlling means.
